(19) United States
(12) Patent Application Publication
(10) Pub. No.: US 2021/0350283 A1
(43) Pub. Date: Nov. 11, 2021

(54) DATA ANALYZER

(71) Applicant: SHIMADZU CORPORATION, Kyoto-shi, Kyoto (JP)

(72) Inventors: Yuichiro FUJITA, Kyoto-shi, Kyoto (JP); Akira NODA, Kyoto-shi, Kyoto (JP)

(73) Assignee: SHIMADZU CORPORATION, Kyoto-shi, Kyoto (JP)

(21) Appl. No.: 17/273,762

(22) PCT Filed: Sep. 13, 2018
(86) PCT No.: PCT/JP2018/034006

§ 371 Date: Mar. 5, 2021

(51) Int. Cl. G06N 20/10; G06N 20/20

(52) U.S. Cl. CPC G06N 20/10; H01J 49/26; G06N 20/20

(57) ABSTRACT

A series of processes of dividing given labeled teacher data into model construction data and model verification data, constructing a machine learning model using the model construction data, and applying the model to the model verification data to identify (label) a sample is repeated multiple times (S2 to S5). Although the machine learning model to be constructed changes when the model construction data changes, an accurate identification can be made with a high probability. Thus, there is a high possibility that an original label and an identification result do not coincide in a mislabeled sample, resulting in misidentification. If the number of misidentifications is counted for each sample to obtain a misidentification rate, the mislabeled sample is identified based on the misidentification rate since the misidentification rate is relatively high in the mislabeled sample (S6 to S7). In this manner, the identification performance of the machine learning model can be improved by detecting the sample included in the teacher data that is highly likely to be in a mislabeled state with high accuracy.

### TECHNICAL FIELD

The present invention relates to a data analysis device that analyzes data collected by various methods such as data obtained by various analysis devices such as a mass spectrometer, a gas chromatograph (GC), a liquid chromatograph (LC), and a spectroscopic measurement device, and more specifically, relates to the data analysis device that uses supervised learning, which is a technique of machine learning, to identify and label unlabeled data or to predict a label. Sometimes there is a case where the term “machine learning” does not include multivariate analysis, but it is assumed in the present specification that the machine learning includes the multivariate analysis.

### BACKGROUND ART

Machine learning is one of useful techniques to find regularity in a large amount of diverse data and to predict and identify data using the regularity, and application fields of the machine learning have been expanding more and more in recent years. As typical techniques of the machine learning, a support vector machine (SVM), a neural network, a random forest, AdaBoost, deep learning, and the like are well known. In addition, as typical techniques of the multivariate analysis, which is included in the machine learning in a broad sense, Principal Component Analysis (PCA), Independent Component Analysis (ICA), Partial Least Squares (PLS), and the like are well known (see Patent Literature 1 and the like).

The machine learning is roughly divided into supervised learning and unsupervised learning. In a case of identifying the presence or absence of a specific disease based on data collected by an analysis device for a subject, for example, if it is possible to collect a large amount of data in advance for patients suffering from the disease and normal individuals not suffering from the disease, supervised learning using these pieces of data as teacher data can be performed. Recently, attempts have been made in various places to diagnose diseases such as cancer by applying the supervised learning particularly to mass spectrum data acquired by a mass spectrometer.

FIG. 12 is an example of a peak matrix in which mass spectrum data of cancer samples and non-cancer samples are organized as teacher data.

This peak matrix takes a sample in the vertical direction and a peak position (mass-to-charge ratio m/z) in the horizontal direction, and uses a signal intensity value of each peak as a value of an element. Therefore, each element in one row in this peak matrix indicates a signal intensity value of a peak at each mass-to-charge ratio for one sample, and the respective elements in a column indicate signal intensity values of all samples at a mass-to-charge ratio. Here, the samples from sample 1 to sample n−2 correspond to the cancer samples, and each of these samples is labeled with a value of “1” indicating cancer. On the other hand, the samples from Sample n−1 to Sample N correspond to non-cancer samples, and each of these samples is labeled with a value of “0” indicating non-cancer. In this case, the label is a binary label.

When such labeled teacher data is used, it is possible to construct a machine learning model that can discriminate between cancer and non-cancer with high accuracy. However, a label of teacher data itself is incorrect in some cases. In the first place, the determination on cancer and non-cancer (or the presence or absence of other diseases) is based on the diagnosis of a pathologist, and it is practically impossible to eliminate an error as long as human judgment is made. In addition, it is also possible to consider a case where a label is incorrect due to an input error of an operator when a result is input in correspondence with the samples even if the result of the diagnosis of the pathologist is correct. Therefore, it is inevitable that a large number of samples given as teacher data include a small number of mislabeled samples with incorrect labels.

One method to deal with this situation is to make a machine learning algorithm such that high identification performance can be obtained even if some mislabeled samples are included in teacher data. However, when an attempt is made to increase the robustness to the teacher data in a mislabeled state, the identification performance inevitably deteriorates. Thus, a general-purpose machine learning technique that can obtain highly balanced robustness and identification performance has not been realized.

Another method to deal with the inclusion of mislabeled samples is to find and remove the mislabeled samples before constructing a machine learning model, or to relabel the mislabeled samples correctly. A technique for detecting an error in a label given by machine learning is proposed in Non Patent Literature 1. However, conventionally, there is no highly reliable statistical method for determining whether or not a sample given as teacher data is mislabeled. Therefore, whether or not data contains a mislabel is currently determined only by a primitive method of, for example in the case of medical data, checking one by one whether measurement dates and pathologist's diagnosis results coincide with the labels attached to teacher data. Such a method is very labor-intensive and inefficient. Even with this method, it is almost impossible to determine whether a sample is truly mislabeled when the pathologist's diagnosis itself is incorrect.

### CITATION LIST

### Patent Literature

Patent Literature 1: JP 2017-32470 A

### Non Patent Literature

Non Patent Literature 1: Itabashi and two others, “Study on semi-supervised learning by detection of mislabeled data”, IPSJ National Convention Proceedings, issued in Mar. 8, 2010, Vol. 72, No. 2, pp. 463-464

### SUMMARY OF INVENTION

### Technical Problem

The present invention has been made to solve the above problems, and an object of the present invention is to provide a data analysis device capable of constructing a machine learning model having high identification performance by accurately identifying and removing a sample that is highly likely to be in a mislabeled state from a large number of pieces of data given as teacher data or by relabeling the sample.

### Solution to Problem

The present invention made to solve the above problems is a data analysis device that constructs a machine learning model based on pieces of labeled teacher data for a plurality of samples and identifies and labels an unknown sample using the machine learning model, and includes a mislabel detection unit configured to detect a sample in a mislabeled state among the pieces of teacher data. The mislabel detection unit includes:

a) a repetitive identification execution unit configured to repeat a series of processes of constructing a machine learning model using pieces of model construction data, which are selected from the pieces of teacher data or are pieces of labeled data different from the pieces of teacher data, and applying the constructed machine learning model to a piece of model verification data selected from the pieces of teacher data to identify and label the piece of model verification data, a plurality of times; and

b) a mislabel determination unit configured to obtain a number of misidentifications in which a label as an identification result and a label originally given to data do not coincide for each sample when the repetitive identification execution unit repeats the series of processes the plurality of times, and to determine whether or not the sample is in the mislabeled state based on the number of misidentifications or a probability of the misidentifications.

In the data analysis device according to the present invention, machine learning includes multivariate analysis in which so-called supervised learning is performed. In addition, a content and a type of data to be analyzed are not particularly limited in the data analysis device according to the present invention, but typically, analysis data or measurement data collected by various analysis devices can be used. Specifically, mass spectrum data obtained by a mass spectrometer, chromatogram data obtained by GC or LC, absorption spectrum data obtained by a spectroscopic measurement device, data obtained by DNA microarray analysis, or the like can be used. Of course, data collected by various other techniques can be used.

In the data analysis device according to the present invention, the machine learning model is constructed based on pieces of labeled teacher data for a plurality of (usually an extremely large number of) given samples. Prior to the construction, the mislabel detection unit detects a mislabeled sample data with an incorrect label among the pieces of given teacher data. That is, the repetitive identification execution unit appropriately selects model construction data and model verification data from the pieces of given teacher data, and constructs a temporary machine learning model using the former data. Then, data of each sample selected as the model verification data is identified and labeled by applying the temporary machine learning model to the latter data. Note that the model construction data is not necessarily data included in the pieces of given teacher data (that is, the data to be determined whether or not it is in the mislabeled state), and may be completely different labeled data. In addition, the model construction data and the model verification data may partially overlap each other or may be exactly the same. Therefore, all of the pieces of given teacher data may be used as the model construction data and model verification data.

For example, if a sample that is truly cancerous but labeled as non-cancer (that is, a sample in the mislabeled state) is identified by a certain machine learning model, this sample should be identified as having cancer in many cases. However, since the label attached to the sample is the non-cancer label, it can be said that this is a misidentification in the sense that a label as the identification result and the original label do not coincide. On the other hand, when a sample with a correct label is identified by the same machine learning model, a label as an identification result and the original label coincide and a correct identification is made in many cases. When there is only one machine learning model, it is virtually impossible to determine with high accuracy whether an original label is correct and a misidentification is made or the identification itself is correct but the original label is incorrect even if the label of a certain sample and a label as an identification result do not coincide and it is determined that the misidentification is made. Stochastically speaking, however, the possibility that the misidentification occurs in the case of the mislabeled state is higher. Thus, if an attempt is made to identify the same sample using different machine learning models and count the number of misidentifications, the number of misidentifications should be large regarding a sample in the mislabeled state while the number of misidentifications should be low regarding a sample with a correct label.

Therefore, the repetitive identification execution unit repeats the above-described series of processes a plurality of times, for example, for pieces of the model construction data which are not the same. Even if a machine learning technique itself is the same, the machine learning model changes when the model construction data changes, and thus, the identification using the plurality of different machine learning models is repeated. The mislabel determination unit obtains the number of misidentifications at the time of repeating such a series of processes a plurality of times, for each sample. That is, the number of misidentifications for the same sample is counted. Since the number of misidentifications is relatively large regarding the sample in the mislabeled state as described above, the mislabel determination unit determines whether or not data of each sample is in the mislabeled state based on the counted number of misidentifications or a misidentification rate obtained from the number of misidentifications. Since it is necessary to determine whether the number of misidentifications is relatively large or small or the misidentification rate is relatively high or low for each sample, as a matter of course, it is necessary to increase the number of repetitions of the above-described series of processes to a certain extent sufficient for this determination.

As described above, the mislabel detection unit can detect data of a sample that is highly likely to be mislabeled among the pieces of teacher data derived from a large number of cancer samples in the data analysis device according to the present invention. Therefore, it is possible to improve the identification performance of the machine learning model constructed using the teacher data by excluding the sample detected in this manner from the teacher data and improving the quality of the teacher data. In addition, if the label is a binary label such as cancer and non-cancer, it is easy to change the label, and thus, data of a sample that has been identified to be highly likely to be in the mislabeled state may be relabeled to remain as the teacher data without being excluded.

In the data analysis device according to the present invention, preferably, the mislabel detection unit is configured to perform processing of the repetitive identification execution unit and the mislabel determination unit at least once using pieces of teacher data obtained after removing the sample determined to be in the mislabeled state by the mislabel determination unit from the pieces of teacher data.

When the sample in the mislabeled state is removed from the teacher data, the identification performance of the machine learning model constructed using the teacher data after the removal is improved. Therefore, this configuration enables the determination with high reliability even regarding data for which it is difficult to determine whether or not data of the sample is in the mislabeled state. As a result, the accuracy of mislabel detection can be improved.

In addition, the model construction data is not necessarily the teacher data to be determined whether or not it is in the mislabeled state as described above in the data analysis device according to the present invention, but it is preferable to select the model construction data from the pieces of teacher data in practical use.

Therefore, as one aspect of the data analysis device according to the present invention,

it is possible to adopt a configuration in which the mislabel detection unit includes a data division unit configured to divide the pieces of teacher data into model construction data and model verification data, and

the repetitive identification execution unit changes the data division by the data division unit each time the series of processes is executed.

In this case, specifically, the data division unit may randomly divide the pieces of teacher data into the model construction data and the model verification data by using, for example, a random number table. Note that, in this case, each piece of data is likely to be the same as data before the change or data after having already been subjected to the process of performing the identification with a low probability even if the model construction data and the model verification data are divided again, but an effect of the possibility hardly appears if the number of repetitions is large.

In addition, the repetitive identification execution unit may be configured to use only one type of machine learning technique or may be configured to use two or more types of machine learning techniques in the data analysis device according to the present invention. As a matter of course, if two or more types of machine learning techniques are used, the configuration of the device (substantially a program for arithmetic processing) becomes complicated, but the accuracy of mislabel detection can be improved by appropriately combining different techniques. On the other hand, even if there is only one type of machine learning technique, the accuracy of mislabel detection can be improved by increasing the number of repetitions.

In addition, in the data analysis device according to the present invention, the machine learning technique used in the repetitive identification execution unit is not particularly limited as long as supervised learning is performed, and a random forest, a support vector machine, a neural network, a linear discrimination method, a non-linear discrimination method, or the like may be used, for example. It is preferable to appropriately select what kind of technique is used depending on a type and properties of data to be analyzed. For example, according to the study of the present inventor, it has been confirmed that the mislabel detection accuracy is relatively high if a random forest is used in a case of identifying whether a subject is cancerous or non-cancerous based on mass spectrum data obtained by mass spectrometry.

In addition, the mislabeled state can be determined by the mislabel determination unit based on various criteria in the data analysis device according to the present invention. As one aspect, the mislabel determination unit may be configured to determine that a sample having the highest misidentification rate is in the mislabeled state.

In this case, one sample that is most likely to be in the mislabeled state is determined to be in the mislabeled state. Thus, it is preferable to remove a plurality of samples that are highly likely to be in the mislabeled state by repeating the processing of the repetitive identification execution unit and the mislabel determination unit while removing the samples determined to be in the mislabeled state one by one as described above.

As another aspect, the mislabel determination unit may be configured to determine that samples as many as a number specified by a user in descending order of the misidentification rate are in the mislabeled state.

In this configuration, a plurality of samples that are highly likely to be in the mislabeled state can be removed at once, and thus, the processing time can be shortened.

As yet another aspect, the mislabel determination unit may be configured to determine that a sample having the misidentification rate of 100% is in the mislabeled state.

With this configuration, the plurality of samples that are highly likely to be in the mislabeled state can be removed with high reliability.

As yet another aspect, the mislabel determination unit may be configured to determine that a sample whose misidentification rate is equal to or higher than a threshold set by the user is in the mislabeled state.

In addition, when the processing of the repetitive identification execution unit and the mislabel determination unit is repeatedly executed in the data analysis device according to the present invention as described above, the mislabel detection unit may be configured to repeatedly perform the processing of the repetitive identification execution unit and the mislabel determination unit until the misidentification rate becomes equal to or lower than a predetermined threshold.

According to this configuration, it is possible to more reliably detect a sample that is likely to be in the mislabeled state. However, the number of repetitions becomes too large in some cases, and thus, a limit may be set on the number of repetitions or a limit may be set on an execution time to end the processing when the limit is violated even if the misidentification rate does not become equal to or lower than the predetermined threshold.

In addition, the data analysis device according to the present invention may further include a result display processing unit configured to create a table or a graph based on an identification result of the mislabel determination unit and displays the table or graph on a display unit.

Specifically, for example, when the distribution of the number of misidentifications or the misidentification rate for each sample of the entire teacher data is illustrated in the graph, the user can easily determine a criteria for determination of any number of misidentifications or any misidentification rate to be regarded as the sample in the mislabeled state.

### Advantageous Effects of Invention

According to the data analysis device of the present invention, it is possible to automatically determine whether or not the given label of the teacher data is incorrect, and identify the sample that is highly likely to be in the mislabeled state. As a result, the quality of the teacher data is improved, for example, by excluding such a sample from the teacher data or relabeling the sample, and it is possible to construct the machine learning model with higher identification performance than that in the related art and to identify the unknown sample more accurately.

### BRIEF DESCRIPTION OF DRAWINGS

FIG. 1 is a functional block configuration diagram of a cancer/non-cancer identification device, which is an embodiment of a data analysis device according to the present invention.

FIG. 2 is a flowchart of a mislabel detection process in the cancer/non-cancer identification device of the present embodiment.

FIG. 3 is a flowchart of a modification of the mislabel detection process in the cancer/non-cancer identification device of the present embodiment.

FIG. 4 is a schematic view of a teacher data division process in the cancer/non-cancer identification device of the present embodiment.

FIG. 5 is an explanatory view of data used in a simulation to verify the mislabel detection ability of the cancer/non-cancer identification device of the present embodiment.

FIG. 6 is a view illustrating the relationship between signal intensities of two marker peaks in an XOR state and a cancerous or non-cancerous state.

FIG. 7 is a view illustrating a mislabel detection result when linear data is used as simulation data.

FIG. 8 is a view illustrating a mislabel detection result when linear data is used as simulation data.

FIG. 9 is a view illustrating a mislabel detection result when non-linear data is used as simulation data.

FIG. 10 is a view illustrating a mislabel detection result when non-linear data is used as simulation data.

FIG. 11 is a graph illustrating a display example of the mislabel detection result.

FIG. 12 is a view illustrating an example of a peak matrix in which mass spectrum data of cancer samples and non-cancer samples are organized as teacher data.

### DESCRIPTION OF EMBODIMENTS

Hereinafter, a cancer/non-cancer identification device, which is an example of a data analysis device according to the present invention, will be described with reference to the accompanying drawings.

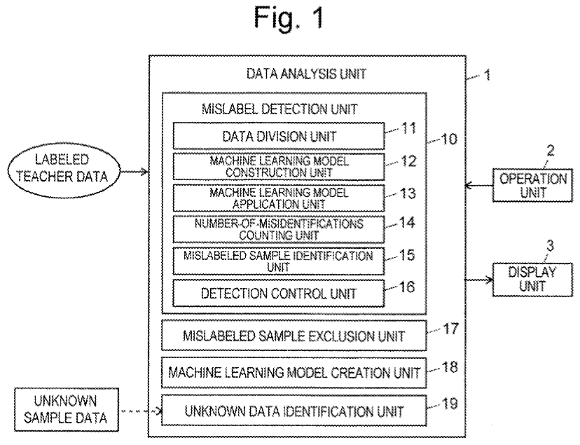

FIG. 1 is a functional block configuration diagram of the cancer/non-cancer identification device of the present embodiment.

This cancer/non-cancer identification device is a device that is used when mass spectrum data obtained by mass spectrometry of a biological sample derived from a subject with a mass spectrometer (not illustrated) is input as unknown sample data to determine whether the sample is cancerous or non-cancerous, and includes a data analysis unit 1, an operation unit 2 and a display unit 3 which are user interfaces.

The data analysis unit 1 includes a mislabel detection unit 10, a mislabeled sample exclusion unit 17, a machine learning model creation unit 18, and an unknown data identification unit 19 as functional blocks. In addition, the mislabel detection unit 10 includes a data division unit 11, a machine learning model construction unit 12, a machine learning model application unit 13, a number-of-misidentifications counting unit 14, a mislabeled sample identification unit 15, and a detection control unit 16 as functional blocks.

Each functional block included in the data analysis unit 1 can be configured by hardware. In practical use, however, it is preferable to adopt a configuration in which each of the above functional blocks is embodied by executing dedicated software installed on a computer on the computer using personal computers and higher-performance workstations as hardware resources.

In the data analysis unit 1, pieces of mass spectrum data derived from a large number of samples labeled as caner or non-cancer as illustrated in FIG. 12 (data indicating a peak signal intensity for each mass-to-charge ratio in which a peak exists) is are in advance as labeled teacher data. The mislabel detection unit 10 detects a sample that is highly likely to be in a mislabeled state from the pieces of given teacher data. The mislabeled sample exclusion unit 17 excludes the sample detected by the mislabel detection unit 10 from the pieces of teacher data, or replaces a label attached to the detected sample. Here, since the label has two values of cancer: 1 and non-cancer: 0, the label can be simply changed from 1 to 0 or from 0 to 1.

The machine learning model creation unit 18 constructs a machine learning model using the teacher data after some samples have been excluded or relabeled by the mislabeled sample exclusion unit 17. A machine learning technique used here may be the same as the machine learning technique used in a mislabel detection unit 10 to be described later, but is not necessarily the same. The unknown data identification unit 19 determines mass spectrum data derived from an unknown sample using the machine learning model constructed by the machine learning model creation unit 18, and gives a label indicating cancer or non-cancer to the unknown sample. Such an identification result is output from the display unit 3.

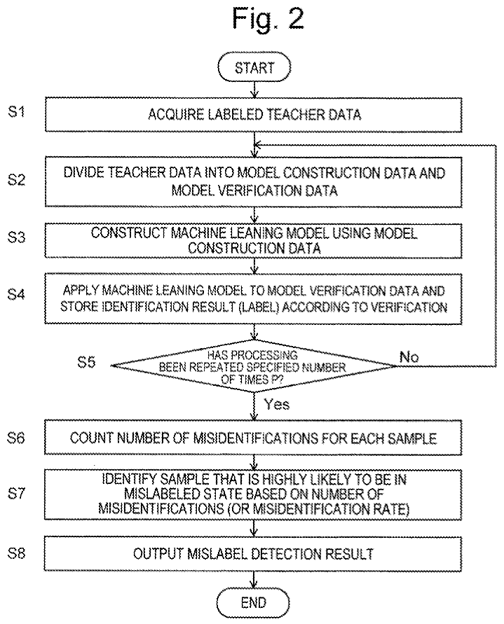

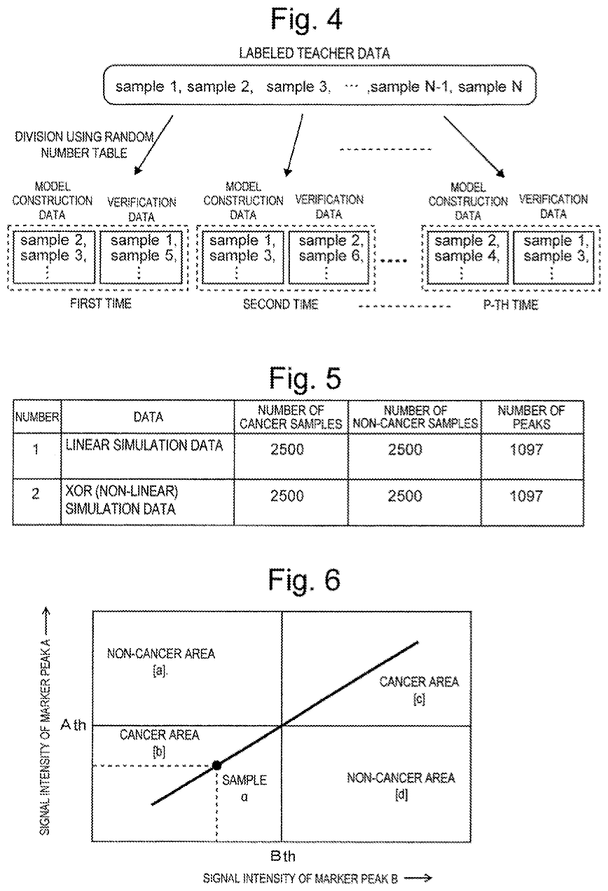

In order for the machine learning model creation unit 18 to construct the machine learning model with high identification performance, it is important to minimize the number of mislabeled samples that are likely to be included in the teacher data. Therefore, in the mislabel detection unit 10 in the cancer/non-cancer identification device of the present embodiment, the sample that is highly likely to be in the mislabeled state is detected with high accuracy by characteristic processing to be described below. FIG. 2 is a flowchart of a mislabel detection process in the cancer/non-cancer identification device of the present embodiment, and FIG. 4 is a schematic view of a labeled teacher data division process.

Under the control of the detection control unit 16, the data division unit 11 reads the labeled teacher data as illustrated in FIG. 12 (Step S1). That is, the labeled teacher data is mass spectrum data of each of N samples having sample names of sample 1, sample 2, . . . , sample N−1, and sample N, and each of the samples is labeled with the binary label of cancer: “1” and non-cancer: “0”. Note that a number of N is preferably large in general, but it is desirable to confirm N in advance since the number of required samples varies depending on the nature of data.

The data division unit 11 divides the pieces of teacher data derived from a large number of read samples into model construction data used to construct a machine learning model, and model verification data to which the constructed machine learning model is applied (Step S2).

Here, pieces of data obtained from the N samples in total are divided into M data sets using a random number table to use M−1 data sets as the model construction data, and the remaining one data set as the model verification data. In this manner, the given teacher data is divided into the model construction data and the model verification data (see FIG. 4). Note that M is set to 5 in simulation verification to be described later.

Since the random number table is used to divide the data, a combination of data contained in a data set may be the same when the division is performed again, but such a probability is extremely low, and the combination of data contained in the data set changes when the division is performed again in many cases.

Next, the machine learning model construction unit 12 constructs the machine learning model by a predetermined technique using the model construction data obtained in Step S2, that is, as the teacher data (Step S3). The machine learning technique used here does not matter as long as the technique is supervised learning. For example, a random forest, a support vector machine, a neural network, a linear discrimination method, a non-linear discrimination method, or the like can be used.

The machine learning model application unit 13 applies the model verification data obtained in Step S2 to the machine learning model constructed in Step S3, and identifies whether each sample is cancerous or non-cancerous to give a label (Step S4). The label given for each sample here is stored, for example, in an internal memory in association with a sample name. Then, the detection control unit 16 determines whether or not the series of processes of Steps S2 to S4 has been repeated a specified number of times P (Step S5), and returns to Step S2 if the number of repetitions has not reached the specified number P.

Returning to Step S2, the data division unit 11 divides the pieces of teacher data derived from a large number of samples into model construction data and model verification data again. At this time, there is a high possibility that a combination of the model construction data and model verification data is different from that of the first time. Even if the machine learning technique is the same, if the model construction data is different, the machine learning model constructed based on the data is also different as a matter of course. Therefore, if the machine learning model different from the previous one is applied to the model verification data, an identification result is likely to be different even if the same sample as the previous one is included in the model verification data. In this manner, the processes of Steps S2 to S5 are repeated the specified number of times P while the division of the teacher data is changed.

As described above and as illustrated in FIG. 4, a combination of samples contained in the model verification data usually changes with each repetition of the above processing, but the same samples are included in the model verification data multiple times if P is increased to some extent, and labeling is performed by the process of Step S4 each time. Therefore, after the number of repetitions of the above series of processes has reached the specified number of times P (Yes in Step S5), the number-of-misidentifications counting unit 14 counts the number of times an originally given label and a label as an identification result do not coincide, that is, the number of misidentifications, for each sample (Step S6). The number of misidentifications is obtained for each sample included in the teacher data read in Step S1.

In the identification based on the machine learning model, there is a possibility that true cancer is determined as non-cancer or true non-cancer is determined as cancer, but such a probability is low. In other words, when the originally given label and the label as the identification result do not coincide, that is, there is a misidentification, it can be said that the possibility that the originally given label is incorrect (in the mislabeled state) is higher than a possibility that the identification itself based on the machine learning model is incorrect. Of course, it is difficult to make such a determination with only one identification result. However, if the number of misidentifications is large when the identification is repeated while the machine learning models are changed, it is reasonable to think that the originally given label is incorrect. Therefore, the mislabeled sample identification unit 15 identifies a sample that is highly likely to be in the mislabeled state based on the number of misidentifications obtained for each sample (Step S7).

However, since the number of times the identification has been executed is not the same for each sample, it is not always appropriate to perform comparison using the number of misidentifications, which is an absolute value. Therefore, it is preferable to calculate a misidentification rate based on the number of times the identification has been executed and the number of misidentifications for each sample, and to identify a sample that is highly likely to be in the mislabeled state based on the misidentification rate.

When it is determined whether or not the sample is in the mislabeled state based on the misidentification rate, one of the following several criteria may be adopted.

(1) One sample having the highest misidentification rate is determined to be in the mislabeled state. However, if there are a plurality of samples having the highest misidentification rate, it may be determined that all of the plurality of samples are in the mislabeled state.

(2) The user specifies the number of samples to be determined to be in the mislabeled state in advance as a parameter using the operation unit 2, and determines that samples as many as the specified number in descending order of the misidentification rate are in the mislabeled state.

(3) Only a sample having a misidentification rate of 100% is determined to be in the mislabeled state. When there are a plurality of samples having the misidentification rate of 100%, all of the plurality of samples may be determined to be in the mislabeled state.

(4) The user specifies a threshold of the misidentification rate for determination as the mislabeled state in advance as a parameter using the operation unit 2, and determines that a sample whose misidentification rate is equal to or higher than the threshold is in the mislabeled state.

Of course, the above (1) to (4) can be combined as appropriate. For example, (1) and (4) may be combined, and a sample having a misidentification rate equal to or higher than a certain threshold and the highest misidentification rate may be determined to be in the mislabeled state. Of course, there may be a case where no sample in the mislabeled state exists in the given teacher data. Therefore, basically, it is reasonable to estimate that a sample having a low misidentification rate is not in the mislabeled state. Conversely, it is reasonable to estimate that a sample having an extremely high misidentification rate is in the mislabeled state.

If the sample in the mislabeled state is identified in this manner, a mislabel detection result and a misidentification detection result may be organized in a table format or a graph format and displayed on the display unit 3, and presented to the user (Step S8).

In addition, the mislabeled sample exclusion unit 17 may exclude the sample determined to be highly likely to be in the mislabeled state as described above from the teacher data or relabel the sample as described above to generate teacher data for constructing a machine learning model to perform an actual identification.

Note that a technique called cross-verification is used in order to reduce a statistical error generally in statistical processing as described above. In the cross-verification in a strict sense, a process of constructing a machine learning model using M−1 data sets out of M data sets as model construction data and applying the remaining one data set to the machine learning model as model verification data to perform an identification is executed M times while the data set selected as the model verification data is changed to calculate, for example, an average value of misidentification rates. On the other hand, the data set divided in Step S2 is processed only once in the processing of the above embodiment, which is different from the cross-verification in a strict sense. However, substantially the same effect as that of the cross-verification can be obtained by repeating the processes of Steps S2 to S5 multiple times while changing samples contained in the data set.

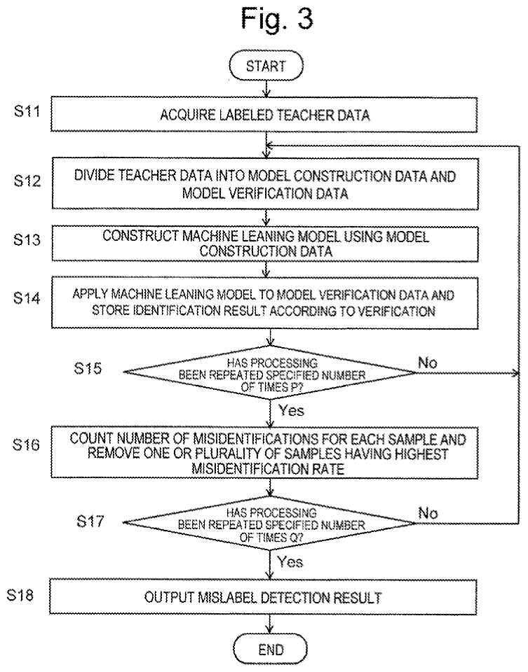

In the mislabel detection process described with reference to FIG. 2, samples that are highly likely to be in the mislabeled state are collectively detected at once after repeating the series of processes of Steps S2 to S4 the specified number of times P, but the flowchart of the mislabeled detection process can be also modified as illustrated in FIG. 3. In FIG. 3, processes of Steps S11 to S15 are exactly the same as the processes of Steps S1 to S5 in FIG. 2.

In this example, after being determined as Yes in Step S15, one or a plurality of samples having the highest misidentification rate obtained for each sample are removed from the teacher data as the samples in the mislabeled state (Step S16). After improving the quality of the teacher data in this manner, the processing returns to Step S12, and the processes of Steps SI2 to S16 are executed again. Then, one or plurality of samples having the highest misidentification rate obtained for each sample are removed from the teacher data again as the samples in the mislabeled state. If the processes of Steps S12 to S16 are repeated a specified number of times Q, or if the highest misidentification rate becomes equal to or lower than a predetermined value or a change of the misidentification rate converges within a predetermined range (Yes in Step S17), the processing is ended.

As the samples that are highly likely to be in the mislabeled state are removed in this stepwise manner, it is possible to further improve the quality of the teacher data more accurately, that is, by removing only the samples that are truly mislabeled while avoiding accidental removal of a non-mislabeled sample.

[Evaluation of Mislabel Detection Process by Simulation]

Next, a result of evaluating whether or not the sample in the mislabeled state is appropriately detected by the above-described mislabel detection process by a simulation will be described. In the evaluation by this simulation, the number M of divisions into the data sets was set to 5 as described above, and the specified number of times P was set to 500. In addition, the random forest was used as the machine learning technique. In addition, as data (teacher data) used for the evaluation, both linear data and non-linear data were used as illustrated in FIG. 5.

[Method and Result of Simulation Using Linear Data]

The linear data referred to herein represents data in which there is a sufficient signal intensity difference in all marker peaks on a mass spectrum between cancer and non-cancer. If the number of marker peaks is large enough and the peak signal intensity difference between cancer and non-cancer is sufficient, the division into two groups of cancer and non-cancer can be performed even by a multivariate analysis technique such as principal component analysis and OPLS-DA (an improved version of partial least squares discriminant analysis (PLS-DA), which is a type of discriminant analysis). Therefore, here, data including 10 marker peaks with almost no signal intensity difference between the cancer and non-cancer was used for the simulation. It has been confirmed that it is impossible to classify the data into two groups even if the principal component analysis is performed.

In addition, since simulation data is known data, a label is 100% valid as a matter f course. Therefore, ten samples were randomly selected from each of cancer and non-cancer samples, and labels of the total of twenty samples were changed to create artificially mislabeled samples. Then, it was verified whether or not these twenty samples could be identified as the mislabeled samples.

In the random forest that uses a decision tree as a learner, a typical parameter that needs to be adjusted is the number of decision trees. When an average correct answer rate in 5-division class verification at the time of changing the number of decision trees was examined, the average correct answer rate was 99.6% regardless of the number of decision trees in the range of five to twenty. Therefore, here, the mislabel detection was tried by setting the number of decision trees to ten.

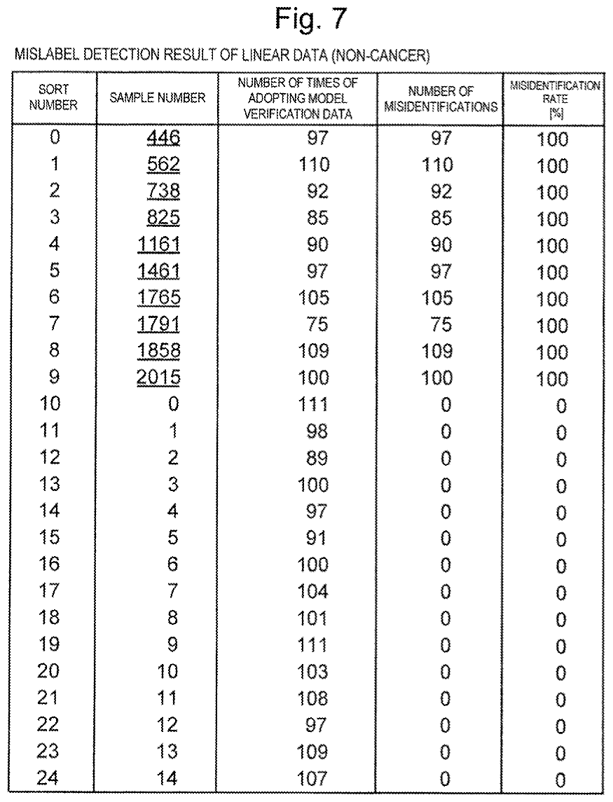

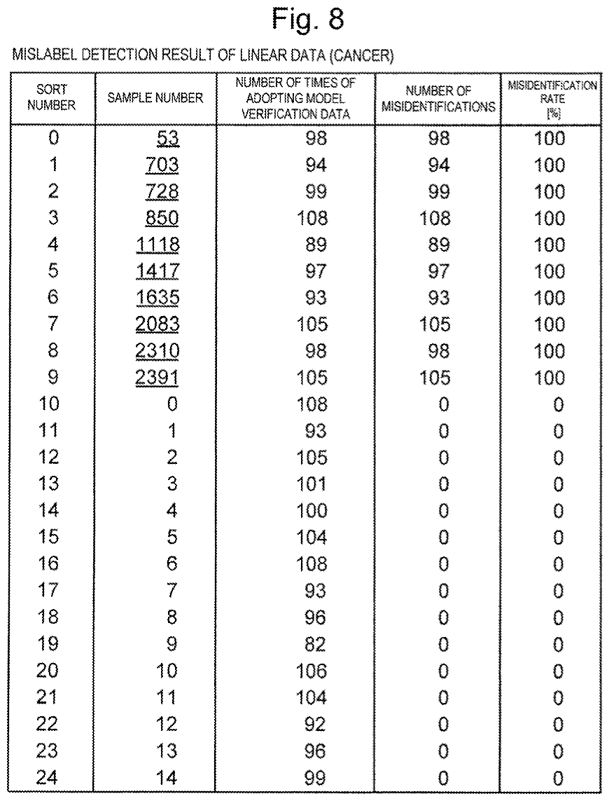

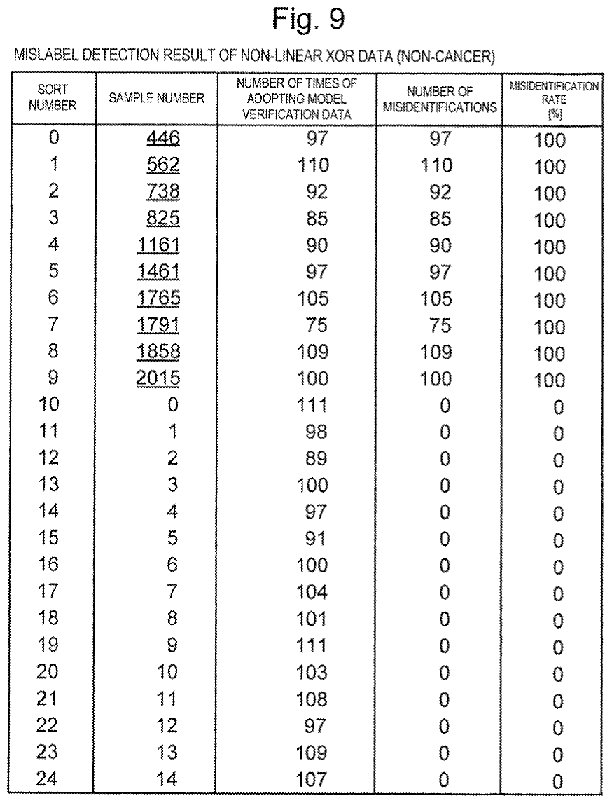

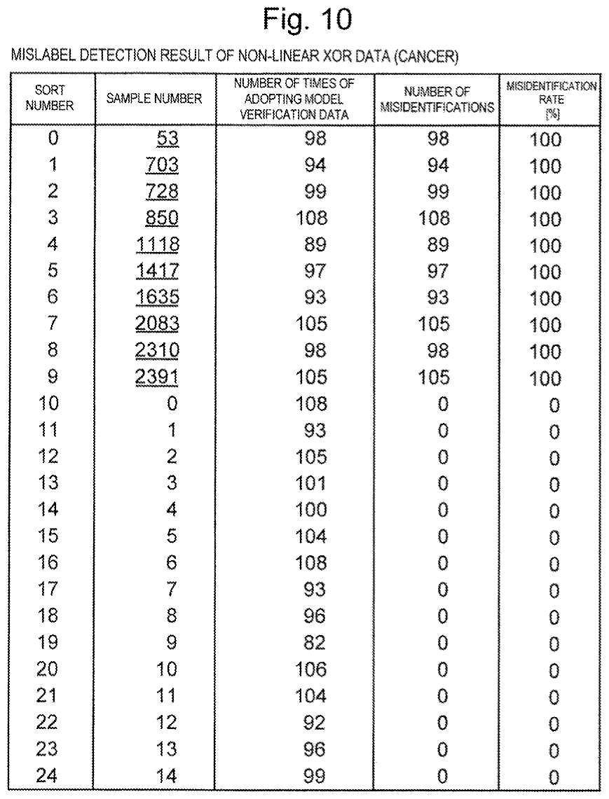

Detection results of the mislabel are illustrated in FIGS. 7 and 8. FIG. 7 illustrates a mislabel detection result of a sample labeled with non-cancer, and FIG. 8 illustrates a mislabel detection result of a sample labeled with cancer. In FIGS. 7 and 8 (and in FIGS. 9 and 10 which will be described later), the number of times of adopting model verification data corresponds to the number of times the identification is executed by the process in Step S4.

As can be seen from FIGS. 7 and 8, a misidentification rate of a mislabeled sample was 100%, and a misidentification rate of a non-mislabeled sample was 0% for both the cancer and non-cancer samples. That is, it can be said that the mislabel detection is completely successful. In addition, in these pieces of data, a correct answer rate for cancer/non-cancer determination in the data including mislabel is 99.6%, but the correct answer rate becomes 100% by removing the mislabeled sample detected by the above-described technique. That is, it can be confirmed that the machine learning model having extremely high identification performance can be constructed by removing the sample identified as the mislabeled sample from the teacher data.

[Method and Result of Simulation Using Non-Linear Data]

Most of data generally collected is not a little non-linear, and rather, few data is perfectly linear. Therefore, the ability of the above-described mislabel detection process was evaluated for non-linear simulation data.

The non-linear data referred to herein represents data that is not capable of identifying cancer or non-cancer from a single peak on a mass spectrum, but can identify cancer or non-cancer by considering a plurality of peaks at the same time. As typical data in such a state, data in which two marker peaks A and B are in an XOR (exclusive OR) state was created. FIG. 6 is a view illustrating the relationship between signal intensities of the two marker peaks in the XOR state and a cancerous or non-cancerous state. That is, it is difficult to identify cancer or non-cancer with each of the two marker peaks A and B alone, but it is determined as cancer (area [c]) if both the signal intensities of the peaks A and B are equal to or higher than thresholds Ath and Bth, respectively, and it is also determined as cancer (area [b]) even if both the signal intensities of the peaks A and B are lower than the thresholds Ath and Bth, respectively. On the other hand, it is determined as non-cancer (area [d]) if the signal intensity of the peak B is equal to or higher than the threshold Bth and the signal intensity of the peak A is lower than the threshold Ath, and it is also determined as non-cancer (area [a]) If the signal intensity of the peak A is equal to or higher than the threshold Ath and the signal intensity of the peak B is lower than the threshold Bth. Therefore, for example, a sample α has cancer.

Artificially mislabeled samples are set to ten samples each for cancer and non-cancer (sample numbers are also exactly the same) similarly to the linear data. In addition, marker peaks with the same mass-to-charge ratio as the linear simulation data were selected, but two peaks among the ten peaks was processed to be in the XOR state each for cancer and non-cancer.

When an average correct answer rate in 5-division class verification at the time of changing the number of decision trees was examined regarding the above data, the average correct answer rate was 99.6% regardless of the number of decision trees in the range of five to twenty. Therefore, the mislabel detection was also tried by setting the number of decision trees to ten here.

Detection results of the mislabel are illustrated in FIGS. 9 and 10. FIG. 9 illustrates a mislabel detection result of a sample labeled with non-cancer, and FIG. 10 illustrates a mislabel detection result of a sample labeled with cancer.

As can be seen from FIGS. 9 and 10, a misidentification rate of a mislabeled sample was 100%, and a misidentification rate of a non-mislabeled sample was 0% for both the cancer and non-cancer samples. That is, it can be said that the mislabel detection is completely successful even in this case. Note that the number of times of adopting the model verification data for each sample is exactly the same between the linear data and the non-linear data, but this is because random numbers in the random number table used for the data division are exactly the same, which does not affect the evaluation results at all.

As apparent from FIGS. 7 to 10, the misidentification rate is 100% for all the mislabel samples, and the misidentification rate is 0% for all the samples with valid labels. This is mainly affected by characteristics of the machine learning technique (random forest) used in this simulation. When the misidentification rates extremely differ between the mislabeled state and the non-mislabeled state in this manner, it is easy to identify the mislabeled sample based on the misidentification rate. Meanwhile, in the case of using another machine learning technique is used, the misidentification rates are not always obtained as above.

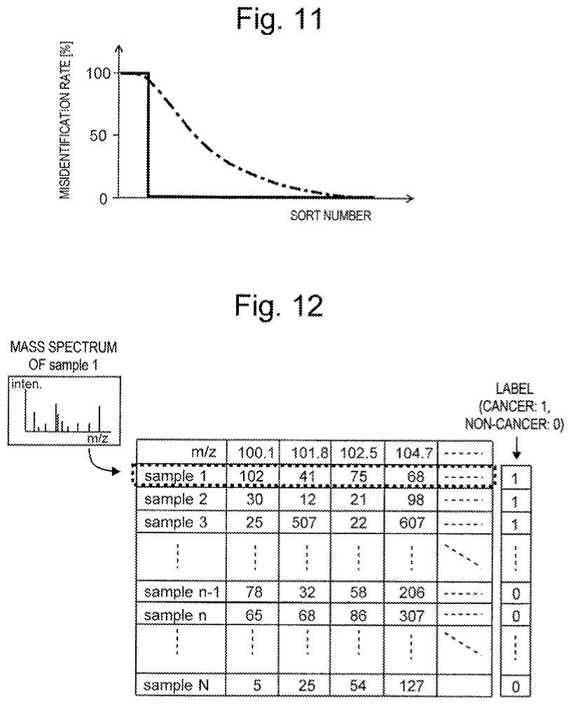

FIG. 11 is a graph illustrating a schematic relationship between sort numbers assigned by sorting the sample numbers in descending order of the misidentification rate and the misidentification rate.

In FIG. 11, a solid line represents the mislabel detection result for the simulation data using the above-described random forest, and an alternate long and short dash line represents an example of a mislabel detection result for simulation data using a support vector machine. In this manner, there is a case where a misidentification rate gradually decreases when the support vector machine is used. In addition, there is a case where the highest misidentification rate does not reach 100%. Therefore, it is advantageous to use a technique of allowing the user to specify a threshold for determining whether or not a sample is in the mislabeled state or removing samples having the highest misidentification rate one by one as illustrated in FIG. 3.

To present the graph as illustrated in FIG. 11 or a table containing the same information to the user is advantageous in terms of allowing the user to select a criterion for determining whether or not the sample is in the mislabeled state, setting a parameter such as the threshold for the determination, and determining whether or not the used machine learning technique is appropriate. Therefore, the graph as illustrated in FIG. 11 or the table corresponding to the graph may be created and displayed on a screen of the display unit 3 after calculating the misidentification rate for each sample in the cancer/non-cancer identification device of the above embodiment.

In the cancer/non-cancer identification device of the above embodiment, the mislabel detection unit 10 uses the random forest as the machine learning technique. However, it is apparent that various supervised learning techniques which have been already exemplified, such as the support vector machine, the neural network, the linear discrimination method, and the non-linear discrimination method, can be used. Since what kind of method is appropriate depends on the nature of data to be analyzed or the like, a plurality of machine learning techniques may be prepared in advance to be arbitrarily selectable by the user.

In addition, at the time of repeating the processes of Steps S2 to S5 in FIG. 2 or repeating the processes of Steps S12 to S15 in FIG. 3, a plurality of types of machine learning techniques may be used, instead of using one type of machine learning technique. Note that it is a matter of course that a machine learning model to be constructed differs for each machine learning technique in the case of using a plurality of different types of machine learning techniques even if the model construction data is the same. Therefore, when performing machine learning by one technique and then performing machine learning by another technique in the case of using the plurality of different types of machine learning techniques, re-division of teacher data may be omitted and the machine learning by the other technique may be performed using the same model construction data and model verification data as those in the case of the machine learning by the one method which has been previously performed.

In addition, the model construction data and the model verification data are always different pieces of data since the pieces of teacher data derived from the sample are divided into the model construction data and model verification data in the above embodiment, but this is not essential. For example, model construction data and model verification data may be arbitrarily selected from a large number of pieces of teacher data (for example, using a random number table). Therefore, the model construction data and the model verification data may be partially common. In addition, the model construction data may be directly used for the model verification data, that is, both of them may be exactly the same.

In addition, the device of the above-described embodiment uses the present invention for the analysis of the mass spectrum data obtained by the mass spectrometer, but it is apparent that the present invention can be applied to all the other devices that performs an identification using machine learning for various types of analysis data and measurement data other than the mass spectrum data. For example, in the field of analysis devices similar to the mass spectrometer, it is apparent that the present invention can be used as a device that analyzes chromatogram data obtained by an LC device or a GC device, absorption spectrum data obtained by a spectroscopic measurement device, or the like. Furthermore, the present invention can also be used for analysis of data (data obtained by digitizing an image) obtained by DNA microarray analysis.

Furthermore, it is a matter of course that the present invention can be used for a data analysis device that performs an identification (labeling) by machine learning based on data collected by various other techniques as well as machine learning based on the data obtained by such device analysis.

That is, the above embodiment is merely an example of the present invention. Any change, modification, addition, or the like appropriately made within the spirit of the present invention from any viewpoints other than the previously described ones will naturally fall within the scope of claims of the present patent application.

### REFERENCE SIGNS LIST

1 . . . Data Analysis Unit
10 . . . Mislabel Detection Unit
11 . . . Data Division Unit
12 . . . Machine Learning Model Construction Unit
13 . . . Machine Learning Model Application Unit
14 . . . Number-of-Misidentifications Counting Unit
15 . . . Mislabeled Sample Identification Unit
16 . . . Detection Control Unit
17 . . . Mislabeled Sample Exclusion Unit
18 . . . Machine Learning Model Creation Unit
19 . . . Unknown Data Identification Unit
2 . . . Operation Unit
3 . . . Display Unit

1. A data analysis device that constructs a machine learning model based on pieces of labeled teacher data for a plurality of samples and identifies and labels an unknown sample using the machine learning model, the data analysis device comprising
a mislabel detection unit configured to detect a sample in a mislabeled state among the pieces of teacher data,
wherein the mislabel detection unit includes:
a) a repetitive identification execution unit configured to repeat a series of processes of constructing a machine learning model using pieces of model construction data, which are selected from the pieces of teacher data or are pieces of labeled data different from the pieces of teacher data, and applying the constructed machine learning model to a piece of model verification data selected from the pieces of teacher data to identify and label the piece of model verification data, a plurality of times; and
b) a mislabel determination unit configured to obtain a number of misidentifications in which a label as an identification result and a label originally given to data do not coincide for each sample when the repetitive identification execution unit repeats the series of processes the plurality of times, and to determine whether or not the sample is in the mislabeled state based on the number of misidentifications or a probability of the misidentifications.

2. The data analysis device according to claim 1, wherein
the mislabel detection unit performs processing of the repetitive identification execution unit and the mislabel determination unit at least once using pieces of teacher data obtained after removing data of the sample determined to be in the mislabeled state by the mislabel determination unit from the pieces of teacher data.

3. The data analysis device according to claim 1, wherein
the mislabel detection unit includes a data division unit configured to divide the pieces of teacher data into model construction data and model verification data, and
the repetitive identification execution unit changes the data division by the data division unit each time the series of processes is executed.

4. The data analysis device according to claim 1, wherein
the repetitive identification execution unit uses only one type of machine learning technique.

5. The data analysis device according to claim 1, wherein
the repetitive identification execution unit uses two or more types of machine learning techniques.

6. The data analysis device according to claim 1, wherein
the repetitive identification execution unit uses random forest as a machine learning technique.

7. The data analysis device according to claim 1, wherein
the repetitive identification execution unit uses a support vector machine as a machine learning technique.

8. The data analysis device according to claim 1, wherein
the repetitive identification execution unit uses a neural network as a machine learning technique.

9. The data analysis device according to claim 1, wherein
the repetitive identification execution unit uses a linear discrimination method as a machine learning technique.

10. The data analysis device according to claim 1, wherein
the repetitive identification execution unit uses a non-linear discrimination method as a machine learning technique.

11. The data analysis device according to claim 1, wherein
the mislabel determination unit determines that data of a sample having a highest misidentification rate is in the mislabeled state.

12. The data analysis device according to claim 1, wherein
the mislabel determination unit determines that pieces of data of samples as many as a number specified by a user in descending order of a misidentification rate are in the mislabeled state.

13. The data analysis device according to claim 1, wherein
the mislabel determination unit determines that data of a sample having a misidentification rate of 100% is in the mislabeled state.

14. The data analysis device according to claim 1, wherein
the mislabel determination unit determines that data of a sample whose misidentification rate is equal to or higher than a threshold set by a user is in the mislabeled state.

15. The data analysis device according to claim 2, wherein
the mislabel detection unit repeatedly performs the processing of the repetitive identification execution unit and the mislabel determination unit until a misidentification rate becomes equal to or lower than a predetermined threshold.

16. The data analysis device according to claim 1, further comprising
a result display processing unit configured to create a table or a graph based on an identification result of the mislabel determination unit and displays the table or graph on a display unit.
